(19) United States
(12) Patent Application Publication
(10) Pub. No.: US 2024/0161870 A1
(43) Pub. Date: May 16, 2024

(54) ALIGNMENT OF TARGET AND REFERENCE SEQUENCES OF POLYMER UNITS

(71) Applicant: OXFORD NANOPORE TECHNOLOGIES PLC, Oxford (GB)

(72) Inventors: Allan Kenneth Evans, Oxford (GB); Marcus Hudak Stoiber, Oxford (GB); Timothy Lee Massingham, Oxford (GB)

(73) Assignee: Oxford Nanopore Technologies PLC, Oxford (GB)

(21) Appl. No.: 18/282,259

(22) PCT Filed: Mar. 15, 2022
(86) PCT No.: PCT/GB2022/050655

§ 371 Date: Sep. 15, 2023

(30) Foreign Application Priority Data
Mar. 16, 2021 (GB) 2103605.8

(51) Int. Cl. G16B 30/00; G16B 40/10

(52) U.S. Cl. CPC G16B 30/00; G16B 40/10

(57) ABSTRACT

A relationship (30) between a target sequence of polymer units in a target polymer (10) and a reference sequence of polymer units (20) in a reference polymer such as an alignment is determined from a measured target signal (11) comprising signal levels measured by a measurement system from parts of the target polymer (10) ordered along the target sequence. The measured target signal (10) is segmented, and a sequence of target signal symbols (13) is derived, each representing a quantised signal level derived from the signal levels of a respective segment. A sequence of reference signal symbols (23) representing quantised signal levels of a sequence of modelled reference signal levels predicted by a measurement system model to be measured from the reference sequence of the reference polymer (20) by the measurement system is also used. The sequence of target signal symbols (13) is aligned with the sequence of reference signal symbols (23) to derive the relationship (30) between the target sequence and the reference sequence.

This application is a national stage filing under 35 U.S.C. § 371 of international application number PCT/GB2022/050655, filed Mar. 15, 2022, which claims the benefit of United Kingdom application number GB 2103605.8, filed Mar. 16, 2021, each of which is herein incorporated by reference in its entirety.

The present invention relates to the analysis of a target polymer using a measured target signal comprising signal levels measured by a measurement system from parts of a target polymer ordered along a target sequence of polymer units in the target polymer.

There is much development of sensitive measurement systems for measuring target polymers, for example measurement systems that comprise a nanopore, in which case the signal levels may be measured by the measurement system during translocation of the polymer with respect to the nanopore. The polymer may be, for example, a polynucleotide or a protein. Measurement systems are known, for example, from US2019/0154655, which supports the analysis of signal data that has not been basecalled, and from US2017/0233804 that implements a reject signal when a sample being is no longer of interest, both of which are incorporated herein by reference in their entirety. A technique for comparing a known reference and an ‘uncalled’ reference is known Kovaka et al., “Targeted nanopore sequencing by real-time mapping of raw electrical signal with UNCALLED”, Nat Biotechnol (2020). However, this technique probabilistically considers k-mers that could be represented by the signal and then prunes the candidates based on the reference encoded within a Ferragina-Manzini index. The technique is based on k-mers and is considered computationally expensive.

The present invention relates to determination of a relationship between the target sequence and a reference sequence of polymer units, for example an alignment between the target sequence and the reference sequence or a measure of similarity between the target sequence and the reference sequence. Determination of such a relationship is a non-trivial task due to complexity of the target measured signal as a result of the measurement system, and typically requires the use of computer processing to implement a complex process.

There is an important need to determine such relationships between the target sequence and a reference sequence in a speedy manner. For example, a determined alignment may be used to determine whether the target signal represents any part of a reference sequence, and if so, which part. The number of applications is huge. Some examples which are by no means limitative are to determine whether a biological sample contains a virus, to determine whether an environmental sample contains an organism, to separate a multiplexed sample into different “barcodes”, to obtain a fast indication of the polymer currently being measured in order to control the operation of the measurement system, for example to continue measurement or reject the target polymer in favour of measuring another target polymer. In many such applications, minimising the usage of computer resources is important, for example to reduce cost and/or increase throughput or because the analysis is being performed in a remote location.

Some known methods of determining an alignment between the target sequence and a reference sequence are as follows.

The standard technique is to estimate (call) the target sequence of the target polymer from the measured target signal and to align the estimated target sequence with the reference sequence. Conceptually, this is straightforward. Processes for deriving alignments of sequences of polymer units have been well developed, and this stage is fast because of decades of software optimisation and development of algorithmic tricks that can be applied in the discrete symbol space. However, the initial stage of estimation (calling) of the target sequence of the target polymer from the measured target signal requires significant computing resources and time, thereby impacting the cost and availability of the technique. It may involve a model of the measurement system, for example using a machine learning approach, which is tractable, but complex.

Another known technique disclosed for example in Loose et al: Real-time selective sequencing using nanopore technology, Nature methods 13, 751 (2016) is to use a model of the measurement system to derive a signal level for each polymer unit in the reference sequence. In this case, the measured target signal may be analysed using event-detection to segment it into signal levels, which results in approximately one signal level per polymer unit depending on the efficacy of the event detection. Then, an alignment between the target signal levels and the reference signal levels may be derived, for example using a dynamic programming method such as dynamic time-warp.

This has an advantage over the standard technique mentioned above in that a model of the measurement system that derives a signal level (polymer unit to signal level) is generally easier to construct, simpler, and faster to apply than a model of the measurement system that estimates the target sequence of the target polymer (signal level to polymer unit). Another advantage is that this estimation only needs to be applied once to the reference sequence and can be done in advance if the reference sequence is known beforehand, in contrast to the modelling in the standard technique that needs to be performed for every measured target signal).

However, the second known technique has a serious disadvantage that the derivation of an alignment is significantly slower. This is because of the need to align signal levels having a continuous range of possible values rather than polymer units having a relatively small number of possible identities. For example, derivation of an alignment of a few thousand shotgun reads against a reference sequence that is an E coli reference may typically take many days and up to a week with this method, while the equivalent alignment stage in the standard technique can be performed in minutes.

Joshi et al., “QAlign: aligning nanopore reads accurately using current-level modelling”, Bioinformatics, 11 Dec. 2020 discloses a different technique, which the authors call QAlign. QAlign estimates (calls) the target sequence of the target polymer from the measured target signal, like the standard technique above. QAlign then uses modelling of the measurement system, specifically using a 6-mer model, to derive a signal level for each polymer unit in the estimated target sequence and uses the same model to derive a signal level for each polymer unit in the reference sequence. The sequences of target and reference signal levels are each quantised into equally populated quantiles to derive sequences of target and reference signal symbols representing a quantised signal levels. Finally, the sequences of target and reference signal symbols are aligned to derive an alignment between the target sequence and the reference sequence.

Joshi et al. claims that, compared to the standard technique above, QAlign provides robustness against modelling errors in the estimation (calling) of the target sequence of the target polymer from the measured target signal. However, QAlign suffers from the same problems as the standard technique set out above that the initial stage of estimation (calling) of the target sequence of the target polymer from the measured target signal requires significant computing resources and time, thereby impacting the cost and availability of the technique.

It would be desirable to alleviate at least some of these problems with the known techniques.

According to a first aspect of the present invention, there is provided a method of determining a relationship between a target sequence of polymer units in a target polymer and a reference sequence of polymer units, wherein the method comprises: receiving a measured target signal comprising signal levels measured by a measurement system from parts of the target polymer ordered along the target sequence; segmenting the measured target signal into segments and deriving a sequence of target signal symbols, each target signal symbol representing a quantised signal level derived from the signal levels of a respective segment; and using a sequence of reference signal symbols representing quantised signal levels of a sequence of modelled reference signal levels predicted by a measurement system model to be measured from the reference sequence of polymer units by the measurement system, comparing the sequence of target signal symbols with the sequence of reference signal symbols to determine the relationship between the target sequence and the reference sequence.

This method provides for determination of the relationship between the target sequence and the reference sequence using a comparison of sequences of target and reference signal symbols. The comparison step may be performed much quicker and with significantly less computing resource than the second known technique described above in which signal levels having a wide range of possible values are aligned, because the comparison is between sequences of target and reference signal symbols that have a relatively small number of possible identities. For example, in the case that the relationship is an alignment, the comparison may be performed using known tools that operate in a “polymer unit space” (or “base space” in the case of polynucleotides). By way of example, For example, derivation of an alignment of a few thousand shotgun reads against a reference sequence that is an E coli reference takes of the order of minutes, rather than many days as with the second known technique, as mentioned above.

Moreover, this is achieved without the need to use modelling of the measurement system to derive a signal level for each polymer unit in the estimated target sequence. This advantage is achieved by segmenting the measured target signal and deriving a sequence of target signal symbols, where each target signal symbol represents a quantised signal level derived from the signal levels of a respective segment.

Surprisingly, the segmentation and quantisation of the measured signal allows the comparison to be performed in a “measurement space” with a reduced number of symbols, thereby avoiding the need to model the measurement system to convert the signal into a “polymer unit space” and then to model the measurement system again to convert the signal back into the “measurement space” with the reduced number of symbols. It is counter-intuitive that such the underlying target and reference sequences can be compared in this manner without ever deriving an estimate of the target sequence, but this method has been demonstrated to work effectively.

The method uses a sequence of reference signal symbols representing quantised signal levels of a sequence of modelled reference signal levels predicted by a measurement system model to be measured from the reference sequence of polymer units. Thus, the method is based on modelling of measurement system to derive a signal level (polymer unit to signal level), but this is easier to construct, simpler, and faster to apply than a model of the measurement system that estimates the target sequence of the target polymer (signal level to polymer unit). Such a model may be easily trained on relatively small amount of data, so is convenient for new measurement systems, for example measurement systems comprising a nanopore.

Moreover, this estimation in respect of the reference sequence may be performed in advance of the application of the method to a particular measured target signal. In such a case the method is supplied with the pre-derived sequence of reference signal symbols, and so the estimation does not impact on the required computing resources or time taken for processing of the measured target signal.

These advantages make the method suitable for a wide range of applications in some examples being as follows.

The method is suitable for a mobile tool for example for diagnosis or to sample ecosystems, as advance modelling in respect of the reference polymer means that only a small amount of processing is needed in the field. In practical terms, these operations could be performed on a mobile device without the resources needed for basecalling.

The method is particularly suitable for determining the similarity between a target polymer and a reference polymer during translation of the polymer through a nanopore and ejecting the polymer from the nanopore depending upon the measure of similarity, for example if the polymer being measured is not of interest. The polymer is typically ejected from the polymer at a rate faster than the rate at which the polymer is caused to translocate the nanopore during measurement. In this way the measurement process can be speeded up by ejecting a polymer from the nanopore without further measurement for a polymer that has been determined not to be of interest, thereby freeing up the nanopore to measure a subsequent polymer. Such a method is described in U.S. Ser. No. 10/689,697, herein fully incorporated by reference in its entirety. Similarly the method could be applied in real-time for multiplexing.

There are also advantages for data security and privacy in human applications. For example in the case of a target sequence of a target polymer comprising a polynucleotide, e.g. DNA, of an individual, no estimate of that target sequence is derived or needs to be stored.

In some cases, the method may be applied to a reference sequence which is derived from a reference signal measured from a reference polymer. This reference signal may comprise signal levels measured by a measurement system (which may be the same or different from the measurement system used to derive the target sequence) from parts of the reference polymer ordered along the reference sequence. The reference sequence may be measured from all the reference polymer or a region of the reference polymer. In that case, the method may include estimating the reference sequence from the measured reference signal using the measurement system model.

In other cases, the method may be applied to a reference sequence which is stored in a memory. In this case, the reference sequence may be obtained from any suitable source, for example a library. Such a stored reference sequence may be known to be derived from a reference signal measured from a reference polymer. Alternatively, such a stored reference sequence may have an unknown derivation, for example being a consensus from many previous experiments, but may nonetheless be considered as corresponding to a reference polymer of a known type.

In general, the reference sequence of polymer units may correspond to the entirety or a region of a reference polymer.

Similarly, the target sequence may correspond to the entirety or a region of the target polymer.

In some cases, the reference sequence of polymer units may correspond to a region of a reference polymer that is the same polymer as the target polymer.

The method may be repeated with plural reference sequences. In this case, the plural reference sequences may correspond to plural different reference polymers or to different regions of the same reference polymer.

The determined relationship may in general be any relationship between the between the target sequence and the reference sequence.

In one important class of applications, the determined relationship is an alignment between the target sequence and the reference sequence. Such an alignment may, for example, be used to determine if all or part of the reference sequence is present or absent in the target sequence.

In other applications, the determined relationship between the target sequence and the reference sequence may be a measure of similarity between the target sequence and the reference sequence.

According to further aspects of the present invention, there may be provided a computer program that is capable of execution in a computer apparatus to cause the computer apparatus to perform a method corresponding to the first aspect of the present invention, a computer-readable storage medium storing such a computer program, or an analysis apparatus arranged to implement a similar method to the first aspect of the present invention.

To allow better understanding, embodiments of the present invention will now be described by way of non-limitative example with reference to the accompanying drawings, in which:

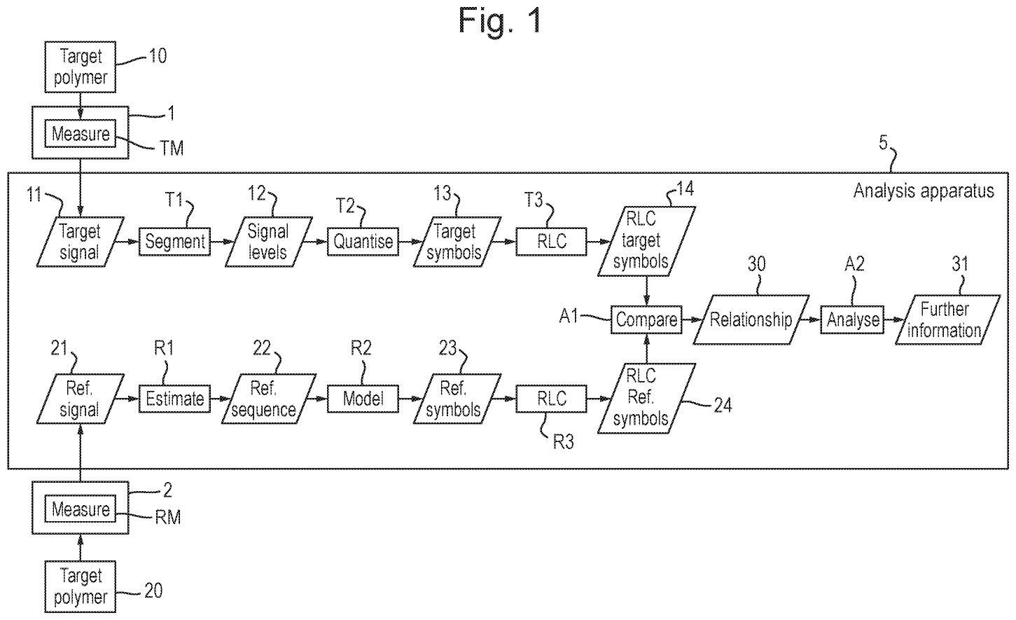

FIG. 1 is a flow chart of a method of determining a relationship between a target sequence and a reference sequence that is performed in an analysis unit;

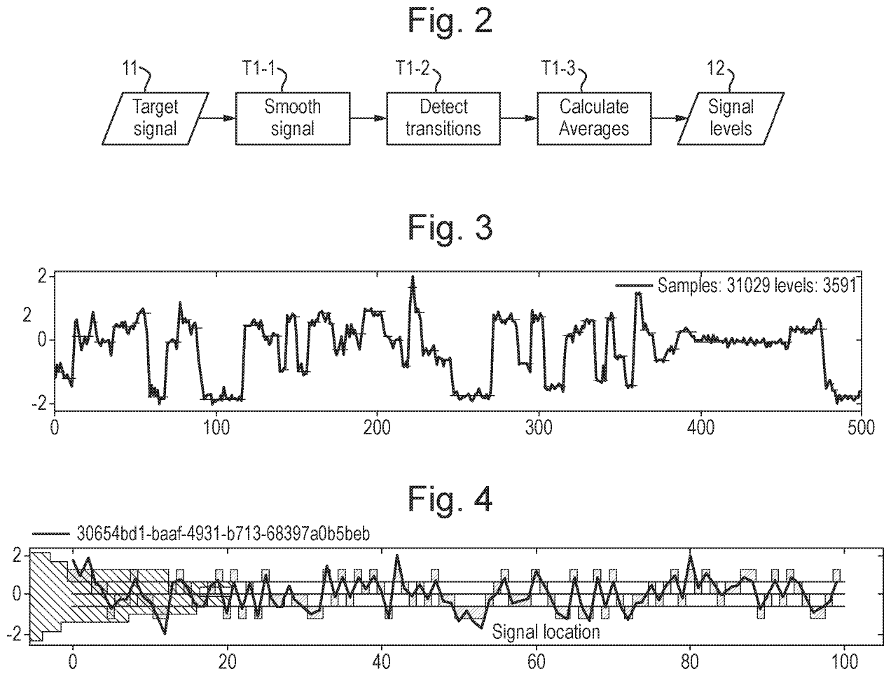

FIG. 2 is a flow chart of an example of a segmenting step of the method of FIG. 1;

FIG. 3 is a plot of an example of a measured target signal showing the results of a segmentation process;

FIG. 4 is a plot of an example of a measured signal showing the derivation of quantiles of the quantised signal levels providing equal populations in each symbol; and

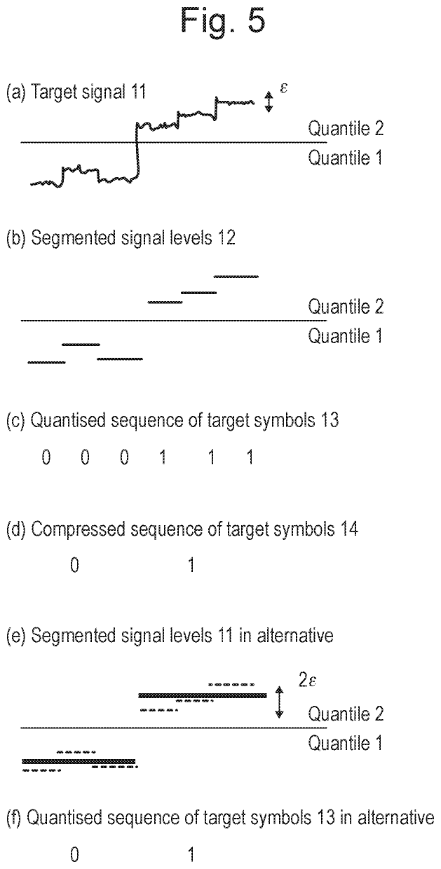

FIG. 5 is a set of diagrams illustrating alternatives for processing the target measured signal.

FIG. 1 illustrates a method of determining a relationship 30 between a target sequence of polymer units in a target polymer 10 and a reference sequence of polymer units 20 in a reference polymer 20. The method is performed as follows.

In step TM, a target measurement system 1 measures a target polymer 10 having a target sequence of polymer units to derive a measured target signal 11. The target measurement system 2 is of a type that sequentially measures signal levels from parts of the target polymer 10 ordered along the target sequence, so the measured target signal 11 comprises a series of signal levels corresponding to successive parts of the target polymer 10. The target signal 11 and the target sequence may correspond to the entirety or a region of the target polymer 10.

The target measurement system 1 may be of any suitable type, some non-limitative examples being as follows.

The target measurement system 1 may comprise a nanopore. In this case, the measured target signal 11 may comprise signal levels measured during translocation of the polymer with respect to the nanopore. This may typically be from parts of the target polymer ordered along the target sequence. The nanopore may be a protein pore or may be a solid state pore. In this case, the target measurement system 1 may be any type of next generation nanopore sequencing apparatus and may measure signal levels representing any one or more of: ionic current, impedance, a tunnelling property, a field effect transistor voltage and an optical property.

The target measurement system 1 may be a sequencing system that uses optical measurements. Examples of such measurements include total internal reflection fluorescence (for example as disclosed in Soni et al., Review of Scientific Instruments 81. 014301 (2010)) and confocal microscopy (for example as disclosed in Fiori et al., “Optoelectronic control of surface charge and translocation dynamics in solid-state nanopores”, Nature Nanotech 8, 946-951 (2013)), and zero-mode waveguide excitation as used in Pacific Biosciences sequencing devices (for example as disclosed in Rhoads et al., “Pacbio sequencing and its applications” Genom. Proteom. Bioinform. 2015; 13:278-289).

The measurement system 1 may be applied to a target polymer in which nucleotides or other polymer units have been systematically substituted by other units to improve the accuracy of the measurement process, as for example in the ‘expandomer’ approach disclosed, for example, in U.S. Pat. No. 7,939,259.

The target measurement system 1 may be any of the types of measurement system disclosed in WO-2020/109773.

The target polymer and reference polymer each comprise a sequence of polymer units and may be any type of polymer that is suitable for measurement in the type of the target measurement system 1. In an important class of applications, the polymer is a polynucleotide, and the polymer units are nucleotides. However, the polymer may be of other types, for example a protein or a polysaccharide. The polymer may be any of the types of polymer disclosed in WO-2020/109773.

The rate of translocation of the polymer through the nanopore may be controlled by various means, such as by control of the potential difference across the nanopore, an enzyme molecular brake, or methods such as disclosed by WO2020016573 and WO2019006214.

Methods for controlling the rate of translocation include, for polymers such as polynucleotides, the use of a polynucleotide binding protein such a helicase, such as described in WO2014013260 and WO2015055981.

The measured target signal 11 output by the target measurement system 1 is supplied to an analysis apparatus 5. The target measurement system 1 may be physically associated with the analysis apparatus 5 or may be located remotely from the analysis apparatus 5. The supply of data may occur over any suitable data connection, for example over a network.

Similarly in step RM, a reference measurement system 2 measures a reference polymer 10 having a target sequence of polymer units to derive a measured reference signal 21. The reference measurement system 2 is of a type that sequentially measures signal levels from parts of the reference polymer 20 ordered along the reference sequence, so the measured reference signal 21 comprises a series of signal levels corresponding to successive parts of the reference polymer 20. The reference signal 21 and the reference sequence may correspond to the entirety or a region of the reference polymer 20.

In some applications, the reference measurement system 2 may be the same type of measurement system, or even the same measurement system, as the target measurement system 1. In other applications, the reference measurement system 2 may be a different type of measurement system from the target measurement system 1. Even when of a different type from the target measurement system 1, the reference measurement system 2 may nonetheless be of any of the types described above for the target measurement system 1.

The measured reference signal 21 output by the reference measurement system 2 is supplied to the analysis apparatus 5. The reference measurement system 2 may be physically associated with the analysis apparatus 5 or may be located remotely from the analysis apparatus 5. The supply of data may occur over any suitable data connection, for example over a network.

That said, step RM is optional and in an alternative implementation, the analysis apparatus 5 is supplied with a measured reference signal 21 that has been measured previously and not as part of the method.

Where step RM is performed at all, typically this is in advance of the step TM of measuring the target polymer 10.

The remaining steps of the method are performed in the analysis apparatus 5 using the measured target signal 11 and the measured reference signal 21 that are received by the analysis apparatus 5. As shown in FIG. 1, steps of the method are performed in functional blocks of the analysis apparatus 5 (shown as rectangles in FIG. 1) having labels with prefixes T (for Target), A (for Analysis) or R (for Reference). As also shown in FIG. 1, the functional blocks process data (shown as parallelograms in FIG. 1) representing various signals and information described in detail below. For example, the relationship 30 is represented by data. Such data may be stored in a storage device of the analysis apparatus 5.

The analysis apparatus 5 may be implemented as a computer apparatus executing a computer program. In this case, the computer program is capable of execution by the computer apparatus and is configured, on execution, to cause the computer apparatus to perform the method including the steps of the functional blocks. Such a computer apparatus may be any type of computer system but is typically of conventional construction. The computer program may be written in any suitable programming language.

The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory. In some embodiments, portions of the computer program may be implemented using hardware amenable to parallelisation of calculations such as a Graphics processing unit (GPU).

Alternatively, analysis apparatus 5 may be implemented by a dedicated hardware device, or by a combination of hardware and software. In such cases, any suitable type of hardware device may be used, for example an FPGA (field programmable gate array) or an ASIC (application specific integrated circuit).

The measured reference signal 21 is processed in the analysis apparatus 5 as follows.

Blocks R1-R3 together form a reference signal processing functional block and operate as follows.

In block R1, the measured reference signal 21 is processed to derive a reference sequence 22 which in this example is an estimate of the reference sequence of the reference polymer 20. This step uses a reference measurement system model of the reference measurement system 2. The model is configured to estimate the sequence from an input signal. Accordingly, the model is used to estimate (call) the reference sequence 22 from the measured reference signal 21.

The block R1 may implement any suitable technique, typically requiring a machine learning technique, for example a neural network. By way of non-limitative example, the block R1 may implement the techniques disclosed in any of WO2013/041878, WO2018/203084, or WO2020/109773.

In some applications, the reference sequence of polymer units may correspond to a region of a reference polymer 20 that is the same polymer as the target polymer 10.

The step performed in block R1 is optional. As an alternative, the analysis apparatus 5 may not use a reference signal 21 at all, and may instead use a reference sequence 22 that is stored in memory. In this case, the reference sequence 22 may have been previously supplied to the analysis apparatus 5. In this case, the reference sequence 22 may have been measured using a reference measurement system 2, but that fact is not used in the method, and the nature of the reference measurement system 2 may not be known. In this alternative, the reference sequence 22 may be taken from any suitable source, such as a sequence library, depending on the application. In particular, the reference sequence 22 does not need to be derived by any measurement system, such as the types of measurement system described above.

In many applications, the reference sequence may not have been derived directly from any single measurement system, but may be the result of cumulative research in the scientific community over a period of time and not derived from a single measurement operation. This is the case for many reference sequences. A good example of this is E. coli. which may be used as a reference sequence, for example to look for evidence of E. coli. infection in a biological sample. A typical E. coli. reference sequence is the result of cumulative research in the scientific community over decades. Nonetheless, in this case the reference sequence may be considered as corresponding to a reference polymer 20 of a known type.

Where the reference signal 21 is received by the analysis apparatus 5 and the step of block R1 is performed, this step is relatively time consuming and requires significantly more computing resource than the analysis of the measured target signal 11 described below, because it is required to resolve different polymer units which may produce similar signal levels.

However, the reference signal 21 is typically received by the analysis apparatus 5 in advance of the analysis of the measured target signal 11, and the step of block R1 may similarly be performed in advance to derive the reference sequence 22 just once for use with repeated instances of the target signal 11. As such, the performance of the step of block R1 does not impact the analysis of the measured target signal 11.

In block R2, the reference sequence 22 is processed to derive a sequence of reference signal symbols 23. This step uses a target measurement system model of the target measurement system 1. The model is configured to derive quantised signal levels that are predicted by the target measurement system model to be measured from the reference sequence 22, if it had been notionally been measured by the target measurement system 1.

It is noted in particular that the model used block R2 models the target measurement system 1 which is different from the reference measurement system 2 modelled in block R1, except of course in the case discussed above that the target measurement system 1 and the reference measurement system 2 are of the same type.

Aside the quantisation of the output signal levels, the model used the step of block R2 is conceptually similar to the model used in the step of block R1. However, it is significantly easier to construct, is simpler, and is faster to apply. This is because modelling of signal levels from a sequence of polymer units is intrinsically easier due to the simpler dependence of signal levels on the polymer units.

The quantisation of the reference signal symbols 23 is the same as the quantisation used in the analysis of the target signal 11 and is discussed further below.

The step performed in block R2 is optional. As an alternative, the analysis apparatus may not use a reference sequence 22 at all, and may instead use a stored signal as the sequence of reference symbols 23. In this alternative, the sequence of reference symbols 23 may have been derived elsewhere and supplied to the analysis apparatus 5.

However, when used, the reference signal 21 or reference sequence 22 are typically received by the analysis apparatus 5 in advance of the analysis of the measured target signal 11, and the step performed in block R2 may similarly be performed in advance to derive the reference sequence 22 just once for use with repeated instances of the target signal 11. As such, the performance of the step of block R2 does not impact the analysis of the measured target signal 11.

In block R3, the sequence of reference signal symbols 23 are run-length compressed to provide a compressed sequence of reference signal symbols 24 (although this is optional as discussed further below).

The run-length compression (RLC) of the reference signal symbols 23 is the same as the run-length compression used in the analysis of the target signal 11 and is discussed further below.

In overview, therefore, the compressed sequence of reference signal symbols 24 represent quantised signal levels of a sequence of modelled reference signal levels predicted by a target measurement system model implemented in block R2 to be measured by the target measurement system 1 from the reference sequence of the reference polymer 20. This compressed sequence of reference signal symbols 24 is used in a comparison process in block A1 as discussed below.

To derive a signal in respect of the target sequence of the target polymer 10 to be compared with this reference, the measured target signal 11 is processed in the analysis apparatus 5 as will now be described. In overview, the target measured signal 11 is used without applying a model of the target measurement system 1, in contrast to the processing of the reference sequence where a model of the target measurement system 1 may be implemented in block R2 to estimate the reference signal symbols 23. In other words, the sequence of the target polymer is not explicitly identified. While known alignment techniques involve basecalling (i.e. derivation of an estimated sequence from a signal) prior to alignment, which is computationally expensive because it requires a basecalling model to be established (e.g. the Q-align method uses a 6-mer model), the present method taught herein does not derive an estimated sequence from the target signal 11 prior to comparison with the reference, thereby reducing the computational complexity.

Blocks T1-T3 together form a target signal processing functional block and operate as follows.

In block T1, the measured target signal 11 is segmented into a series of segments to derive a series of signal levels 12 in respect of the segments.

FIG. 2 illustrates an example of block T1 in which the segmentation is performed by detecting segments of similar values by identifying transitions in the signal level, as follows. In block T1-1, the measured target signal 11 is smoothed. The purpose is to remove noise that could falsely be detected as a transition. Any suitable smoothing technique may be used. In the simplest case, the smoothing could use a linear filter. In one example, the smoothing is performed by total-variation de-noising. Total-variation denoising is a well-known method. A suitable, fast algorithm for total-variation de-noising is disclosed in Condat, “A Direct Algorithm for 1D Total Variation Denoising”, 2012, hal-00675043v1.

Other common approaches include median filtering and bilateral filtering.

In block T1-2, the smoothed measured target signal 11 is processed to detect transitions in the signal level of the smoothed measured target signal 11, the measured target signal 11 being segmented into segments defined between the transitions. This may be done by detecting discrete levels within the signal. The simplest method applies a threshold for a step to a new level. Another approach is to apply a statistic like a t-test to decide whether a new level should be created. In general, it is possible to apply techniques that have been applied to detect events within measured signals from measurement systems comprising nanopores, on which many variations are known.

In block T1-3, an average signal level is derived from the signal levels of each segment, thereby producing the series of signal levels 12.

FIG. 3 illustrates an example of a measured target signal 11 showing the results of the segmentation process of FIG. 2. In FIG. 3, the series of horizontal lines represent the length and average signal level of the detected segments. As can be seen, the segments correspond to successive portions of the measured target signal 11 having similar values.

With typical measurement systems comprising a nanopore that ratchets the translocation of the polymer with respect to the nanopore, the segments detected by the segmentation process of FIG. 2 may conceptually be considered as corresponding to successive groups of k polymer units (k-mers), where k is a plural integer. In this case, there is approximately one segment per polymer unit, subject to the ability to discriminate between the signals arising from successive k-mers. However, while this is a useful concept for understanding, it may not be an accurate description of all measurement systems and is not necessary or used in the segmentation.

However, FIG. 2 is merely an example and the segmentation step of block T2 could be performed in other ways. In a simple alternative, the segmentation step of block T2 could simply comprise segmentation of the measured target signal 11 into segments of identical length, albeit that would have an impact on the subsequent run-length compression that is described below.

In block T2, the series of signal levels 12 are quantised to derive a sequence of target signal symbols 13. The average signal levels in respect of each segment are quantised. As a result, each target signal symbol represents a quantised signal level derived from the signal levels of a respective segment.

The nature of the quantisation in blocks T2 and R2 is as follows.

Typically the number of symbols is relatively low, for example no more than 10, and preferably no more than 6. In many applications, there may be the same number of symbols as types of polymer unit, for example four symbols in the case that the polymer is a nucleotide and the polymer units are nucleotides (bases) C, G A and T. However, while this is useful conceptually, it is not necessary that there is any connection between the number of symbols and the number of polymer units. Thus, there may be differing numbers and the method may work with a number of symbols as low as two.

In a simple example, the quantisation may be performed with symbols corresponding to bins of equal width, as is the case in a typical analogue to digital converter (ADC). With a typical ADC, there are a large number of symbols (bins) as it is desired to represent any arbitrary signal use. Such an approach works here, but as the number of symbols is much lower there is a risk that some symbols are used significantly more than others. Thus, accuracy can be improved by making more efficient use of bandwidth. Thus, more preferably the quantisation may be performed with symbols corresponding to quantiles of unequal width that are chosen to provide equal populations in each symbol, having regard to the target measured signal 11 itself or to a typical measured signal from the target measurement system 1.

To achieve this, a histogram of the target measured signal 11 itself or to a typical measured signal may be used to select the quantiles with equal population. FIG. 4 illustrates an example of such a measured signal (shifted and scaled on the y-axis so it has median zero and variance of about one) showing the derivation of the quantiles. In FIG. 4, the shading on the left is a histogram of signal levels for a the entire measured signal, the horizontal black lines are boundaries between the quantiles and the shaded blocks show the quantisation of segments into symbols. As can be seen in the example of FIG. 4, if the quantiles were of equal width, then nearly all the data would be in the middle two quantiles.

In block T3, the sequence of target signal symbols 13 are run-length compressed to provide a compressed sequence of target signal symbols 14 (although this is optional as discussed further below).

The run-length compression of blocks R3 and T3 may be performed as follows.

The run-length compression reduces the run length of runs of repeated symbols.

In one approach, each run of repeated symbols may be compressed to a single symbol. As an example of this approach, a sequence of symbols ACCCCGTTTG becomes ACGTG.

In another approach, compression may occur by truncating each run of repeated symbols beyond a predetermined length, for example t symbols, where t is a plural integer, for example being three. As an example of this approach where t=3, a sequence of symbols AAAAACCGTTTTTT becomes AAACCGTTT.

This step increases the accuracy of the subsequent comparison by bringing the number of target signal symbols 14 and reference signal symbols 24 closer to the number of polymer units in the target sequence and reference sequence, respectively. Conceptually, the run-length compression may be thought of as reducing problems caused by the segmentation of step T1 occurring in incorrect locations. This usually happens within a quantile. By applying run-length compression, disagreement with the reference caused by this mis-segmentation is removed.

Blocks A1 and A2 form an analysis functional block and operate as follows.

In block A1, the compressed sequence of target signal symbols 14 is compared with the compressed sequence of reference signal symbols 24 to determine a relationship 30 between the target sequence and the reference sequence.

The relationship 30 that is determined in block A1 may in general be any relationship between the between the target sequence and the reference sequence. As mentioned below, the relationship 30 may, for example, be one that allows subsequent determination, as between the target sequence and the reference sequence, of any one or more of: a match; a difference; a degree of similarity; a degree of difference; and a level of association. The latter case of a level of association may, for example, be one using a threshold level.

In one important class of applications, the relationship 30 is an alignment between the target sequence and the reference sequence. Such an alignment comprises a mapping between the polymer units of the target sequence and the polymer units of the reference sequence. Such an alignment may further comprise a score representing the quality of the mapping. Such a quality score may be a measure of similarity. In some cases, the alignment may comprise plural different mappings with respective quality scores.

In this case, the comparison performed in block A1 may be an alignment process using known tools that operate in a “polymer unit space” (or “base space” in the case of polynucleotides). One example of a suitable tool for performing the alignment is Minimap2 as disclosed in Li, “Minimap2: pairwise alignment for nucleotide sequences”, Bioinformatics, 34(18), 15 Sep. 2018, 3094-3100 (2018). Many other suitable tools also exist, for example LAST disclosed in Kielbasa et al., “Adaptive seeds tame genomic sequence comparison”, Genome research 21(3), 487 (2011).

In some applications, the determined relationship between the target sequence and the reference sequence may be a measure of similarity between the target sequence and the reference sequence. Such a measure of similarity may be a score that does not indicate the mapping between the polymer units of the target sequence and the polymer units of the reference sequence. In this case, the comparison performed in block A1 may be performed using tools that do not attempt to provide an alignment between two sequences but merely provide a measure of similarity or subsequence similarity. An example is BLAST as disclosed in Altschul et al. “Basic local alignment search tool”, Journal of Molecular Biology. 215 (3), 403 (1990).

In this context, the term “measure of similarity” is used to encompass measures that increase with increasing similarity and measures that increase with increasing difference between the target sequence and the reference sequence (which may also be referred to as measures of difference).

As the comparison is being performed in “signal space” but with a relatively small set of possible symbols, such a comparison may be performed at high speed and with relatively few computing resources compared to attempting to compare the underlying signals themselves. However, this is achieved without the need to model the measurement system to convert the signal into a “polymer unit space” and then to model the measurement system again to convert the signal back into the “measurement space” with the reduced number of symbols. It is surprising that the segmentation allows the comparison to provide an accurate determination of the relationship between the target sequence and the reference sequence, but results show this to be possible.

In block A2, the relationship 30 output from the comparison performed in block A1 may be analysed to derive further information 31 about the relationship between the target sequence and the reference sequence. By way of non-limitative example, the analysis in block A2 can determine, as between the target sequence and the reference sequence, of any one or more of: a match; a difference; a degree of similarity; a degree of difference; and a level of association. The latter case of a level of association may use, for example, a threshold level.

Depending on the application, the determined relationship 30 may have a number of uses.

One option shown in FIG. 1, which is applicable where the determined relationship is an alignment between the target sequence and the reference sequence, is that the further information 31 derived in block A2 from the determined relationship 30 is whether all or part of the reference sequence 22 is present or absent in the target sequence.

In some applications, the method shown in FIG. 1 may be repeated with plural reference sequences 22. The plural reference sequences may, for example, correspond to plural different reference polymers 20 or to different regions of the same reference polymer 20.

In the case of plural reference sequences 22, the further information 31 derived in block A2 from the determined relationship 30 may be whether all or part of any of the reference sequences 22 is present or absent in the target sequence. By way of example, after the target symbols 13 or RLC target symbols are identified that can be compared, respectively, with the reference symbols 23 or the RLC reference symbols 24 the method can determine whether they match using the analysis A2. If they do not match, the target symbols 13, 14 can be compared with another set of reference symbols 23, 24 and the process repeated.

The level of analysis in block A2 can be made at a high-order level. For example, where the target polymer has been obtained from a sample of meat, and a plurality of reference polymers have been derived from different animals, and the further information 31 may be the type of animal from which the meat originated.

Analysis at a mid-level can involve obtaining reference symbols from a reference polymer of a virus, such as severe acute respiratory syndrome coronavirus 2 (SARS-CoV-2), and determining a match with target symbols 13 obtained from a sample, such as a blood sample.

The analysis in block A2 can be performed to provide the further information 31 that is the identity of the presence of specific components within target symbols obtained from a target polymer. For example, the reference symbols can include sub-sets of symbols from a plurality of reference polymers. A sub-set of symbols can include, for example, a sequence of polynucleotides of interest, which can include canonical and non-canonical bases. A sub-set can include reference symbols that represent, for example, the presence of

Techniques using tools such as Minimap can speed up the analysis process, wherein all k-mers in the reference are indexed.

Depending on the application, the nature of the target polymer 10, the nature of the reference polymer 20 and a match detected in block A2 may vary. Some non-limitative examples of applications and the consequent nature of the target polymer 10, nature of the reference polymer 20 and match detected in block A2 are shown in Table 1.

TABLE 1

| Application & | Reference | | |
|---|---|---|---|
| target polymer 1 | Polymer 2 | Match required | Note |
| DNA Barcoding | Additional | Start of target | |
| | nucleotide | & | |
| | sequence | All of reference | |
| | added to | | |
| | sample, used | | |
| | to identify | | |
| | source | | |
| Non-DNA | Additional | Start of target | |
| barcoding | non-DNA | & | |
| | polymer | All of reference | |
| | segment added | | |
| | to sample, | | |
| | used to | | |
| | identify source | | |
| Ecosystem or | Multiple | All of target | Applications in remote |
| community | references for | & | environments benefit from |
| profiling | different | Some of any | low computational cost |
| | organisms | reference | |
| Pathogen | Multiple | All of target | |
| identification | references for | & | |
| | different | Some of any | |
| | organisms | reference | |
| Information storage | Code | Part of target | |
| in DNA (retrieval | sequences (e.g. | & | |
| using this method) | two easily | All of reference | |
| | distinguished | | |
| | segments | | |
| | representing | | |
| | bits 0 and 1) | | |
| Identifying | Multiple | Part of target | May use cumulative measure |
| damaged or | references for | & | of evidence from multiple |
| corrupted | different | Part of reference | fragments, if enough separate |
| biological samples | organisms | | examples of small parts of a |
| (e.g. ancient DNA, | | | genome are available |
| forensic samples) | | | |
| Identifying genetic | Different | Part of target | Compare match between two |
| variants | references for | & | possible references. Many |
| | different | All of reference | examples may be needed to |
| | genetic | | gather enough evidence |
| | variants | | |
| Identifying | Different | Part of target | Compare match between two |
| epigenetic changes | references for | & | possible references. Many |
| (methylation of | modified/ | All of reference | examples may be needed to |
| DNA etc) | unmodified | | gather enough evidence |
| | DNA segments | | |
| Counting (near-) | Reference for | Part of target, | |
| repeats (e.g. | repeat segment | containing | |
| tandem repeats | | multiple copies of | |
| used in DNA | | reference | |
| profiling, repeat | | | |
| counts of short | | | |
| segments which are | | | |
| characteristic of | | | |
| Huntington's | | | |
| disease, Friedreich | | | |
| ataxia etc. | | | |
| ‘Read-until’ - that is | Reference for | Varies according | |
| control of | small part of | to application | |
| operation of | desired or | | |
| measurement | rejected | | |
| system | samples | | |

Numerous variations to the method shown in FIG. 1 and described above are possible. Some non-limitative examples of possible variations are as follows, which may be applied in any combination.

A first possible variation is as follows. In the step performed by block A1, the comparison of the compressed sequence of target signal symbols 14 with the compressed sequence of reference signal symbols 24 to be performed using a weight matrix that considers differences between the quantised levels represented by the target signal symbols 14 and the reference signal symbols 24. Use of such a weight matrix may increase accuracy, as follows.

In the absence of using a weight matrix, all mappings where the target signal symbols 14 and the reference signal symbols 24 differ are considered equally bad. For example, suppose that symbols A, C, G, T represent ordinal quantiles (e.g. corresponding to ordinal signal levels 1, 2, 3, 4), then Table 2 shows two mappings that are regarded as equally close, because they both differ at the second location.

TABLE 2

| | Mapping 1 | Mapping 2 |
|---|---|---|
| Reference symbol | CGT | CGT |
| Target symbol | CTT | CAT |
| Reference quantiles | 234 | 234 |
| Target quantiles | 244 | 214 |

However, mapping 1 should be considered as closer in the sense that the differing signal levels of the middle symbol are in the adjacent quantiles (3, 4), while in mapping 2 the differing signal levels of the middle symbol are in in quantiles (3, 1) and so are two quantiles apart. The use of a weight matrix that considers differences between the quantised levels represented by the target signal symbols 14 and the reference signal symbols 24 deals with this issue by weighting mapping 1 as being closer than mapping 2. There are various fast symbol-based mapping tools that may be used with such weight matrix, for example the LAST tool (http://last.cbrc.jp/, as discussed at http://last.cbrc.jp/doc/last-matrices.html).

As noted above, the run-length compression of blocks R3 and T3 is optional in the processing of the target sequence and/or the reference sequence, prior to comparison.

Thus, a second possible variation is to omit the run-length compression of the sequence of reference signal symbols 23 performed in block R3. In this case, the step performed by block A1 is performed on the sequence of reference signal symbols 23 instead of the compressed sequence of reference signal symbols 24.

Similarly, a third possible variation is to omit the run-length compression of the sequence of target signal symbols 13 performed in block T3. In this case, the step performed by block A1 is performed on the sequence of target signal symbols 13 instead of the compressed sequence of target signal symbols 14.

Typically, either the run-length compression of blocks R3 and T3 are both performed or both omitted, although there may be embodiments one of the run-length compression of blocks R3 and T3 is performed and the other is omitted. Run-length compression makes the method more effective in the case where the number of signal levels produced by the segmentation in step T1 is not equal to the number of polymer units in the reference sequence 22. This difference may be, for example, the result of errors in segmentation. It may also occur because the signal level does not change when a polymer unit is repeated, and the time for polymer units to pass through the measurement device is variable. In this case, for it may not be possible for any segmentation algorithm to differentiate between a run of two identical polymer units and a run of three identical polymer units, for example. In cases where the number of signal levels produced by the segmentation in step T1 is known to be equal to the number of polymer units in the reference sequence, run-length compression is not necessary, although it may be used to reduce the length of symbol sequences and so speed up processing.

The run-length compression of the sequence of target signal symbols 13 performed in block T3 is optional and the comparison performed by block A1 may be performed without it. However, the run-length compression of the sequence of target signal symbols 13 may provide some increase in accuracy, depending on the segmentation of the measured target signal 11 performed in block T1. This is because the segmentation and the run-length compression work together to give an output (i.e. the series of target symbols 13), and aim is to match the characteristics of that output to the reference in block A1 (i.e. the series of reference symbols 13 or the compressed series of reference symbols 14).

The run-length compression in block T3 may therefore be considered as being part of the segmentation process, since the outcome is to group a number of signal levels together into a single unit that becomes a quantile symbol. So use of a different segmentation method may remove the need for run-length compression.

A non-limitative example that illustrates this is shown in FIG. 5 and will now be described.

As a comparative example, FIGS. 5(a)-(d) show the processing of the measured target signal 11 in method of FIG. 1 including run-length compression.

FIG. 5(a) shows an example of the measured target signal 11 and the boundary between two quantiles corresponding to symbols and a transition level c used to detect transitions.

FIG. 5(b) shows the series of signal levels 12 produced by the segmentation in block T1 and corresponding to parts of the measured target signal level 11 that differ by more than the transition level E. In this example, the transition level c is equivalent to that selected for event detection in known methods for analysing a measured target signal to identify the sequence of polymer units (e.g. base-calling).

FIG. 5(c) shows the sequence of target symbols 13 obtained by the quantisation in block T2.

FIG. 5(d) shows the compressed sequence of target symbols 14 obtained by the run-length compression in block T3.

FIGS. 5(e) and (f) show the processing of the measured target signal 11 shown in FIG. 5(a) in an alternative without run-length compression.

In this alternative, an increased transition level 2c is used and FIG. 5(e) shows the series of signal levels 12 produced by the segmentation in block T1 and corresponding to parts of the measured target signal level 11 that differ by more than the increased transition level 2c. In this alternative, the transition level 2c is greater than that selected for event detection in known methods for analysing a measured target signal to identify the sequence of polymer units (e.g. base-calling).

It can be seen that the change in the segmentation results in effectively joining together segments that were subsequently compressed together in the run-length compression.

FIG. 5(f) shows the sequence of target symbols 13 obtained by the quantisation in block T2 and is the same as the compressed sequence of target symbols 14 in the comparative example. Thus, in this alternative, the run-length compression in block T3 is unnecessary and so is omitted.

Other changes to the segmentation in block T1 may be performed to achieve a similar effect to the run-length compression. One possibility is for the transition level £ in the segmentation in block T1 itself to be unchanged, and instead to introduce an extra step, prior to the quantisation in block T2, of joining segments whose median levels are less than a predetermined threshold, whose range of signal levels overlap, or whose range of signal levels are separated by less a predetermined threshold. These possibilities may be advantageous to an increase in the transition level £ in the segmentation in block T1, as that intrinsically makes the segmentation less sensitive to signal level variation.

Another situation where the run-length compression in block T3 may be unnecessary and so omitted is that the nature of the target measurement system 1 is that the measured target signal 11 provides a clear boundary between parts of the measured target signal 11 corresponding to different polymer units, so that the segmentation in block T1 may accurately detect those boundaries.

In contrast, in the alternative mentioned above that the segmentation step of block T1 comprises segmentation of the measured target signal 11 into segments of identical length, then the performance of run-length compression in block T3 may be more important.

A fourth possible variation is to combine the segmentation step of block T1 and the quantisation step of T2 to detect groups of signal levels within respective quantiles (desirably with filtering to smooth transitions) and directly output the sequence of target symbols 13. For example, this might involve assigning measured signal levels to quantiles, filtering to remove short spikes, optionally removing runs shorter than 3 samples, and then run-length compression to derive the target symbols 13.

The following method of method of deriving an alignment between a target sequence and a reference sequence was performed for comparison with a comparative example. These methods were performed using a 40-cpu Intel® Xeon® CPU E5-2630 v4 running at 2.20 GHz, which was the test machine used for comparison.

As a test set, the target signal 11 was the raw data for 5000 reads recorded from a test sample of PCR-amplified SCS110 E coli DNA on an ONT Minion device using the R9.41 pore. The reads had been pre-selected by basecalling and mapping the basecall to the E coli chromosome, removing those that did not map. In the raw data, each read comprised a vector of current values, sampled at 4 kHz and the total number of current samples in the reads was 350 million.

SCS110 is a variant of E coli in which the DNA has fewer chemical modifications than other strains, making it particularly suitable for PCR amplification. Samples are commercially available, along with a standard reference nucleotide sequence.

For the comparative example, these reads were basecalled using ONT's Guppy package. Using 40 processor cores in the CPU mode (10 callers, 4 threads per caller), this took 3 hours and 18 minutes on the test machine. This would have been much faster using a GPU, but the purpose of this exercise was to compare timings with the method disclosed herein, which is not yet implemented on a GPU. As mentioned above, the usual method for testing to see whether the reads contain examples of a reference DNA sequence would be to basecall the reads and then perform an alignment or index search of the read sequences against the reference. This time of more than 3 hours therefore provides a lower limit on the time needed for such methods.

The basecalls were then mapped to the SCS110 E coli chromosome reference using minimap2, which took of the order of a minute. The estimated start and end locations of each read on the chromosome according to this method were recorded.

The method shown in FIG. 1 was then tested for the same target signal 11 and reference sequence 22 (i.e. steps RM and R1 were not necessary and not performed).

In these examples, the quantisation process applied in steps T1 and R2 has as its input a vector of numbers, and as its output a list of letters which has the same length as the input. The quantisation procedure had the following steps:
1. Calculate three quantile boundaries q1, q2, q3 for the input vector. The quantile boundaries are defined so that one-quarter of the data points have values less than q1, one-quarter have values v such that q1<=v<q2, one-quarter have values q2<=v<q3 and one-quarter have values v>=q3.
2. Replace each number in the input vector by its quantile number: so numbers less than q1 become 1, numbers in the range (q1, q2) become 2, and so on.
3. Replace the quantile numbers by base letters, using the code 1->A, 2->C, 3->G, 4->T

For use in step R2, a neural-network model of the pore levels was trained on PCR DNA data, to the SCS110 E coli reference sequence. The model was applied in step R2 and an output of this model was a vector of estimated current levels, with one level for each base in the reference sequence. The level vector was quantised using the procedure given above to provide the sequence of reference symbols 23, which was run-length compressed in step 23 to provide the compressed sequence of reference symbols 24.

Because some of the reads in the sample were expected to be reverse-complemented with respect to the E coli reference, we also created a separate reference symbol sequence using the same method, but starting with the reverse-complemented E coli reference.

The production of the compressed sequence of reference symbols 24 from the E coli reference sequence 22 took 61 seconds using a single processor core on the test machine. The speed of this could be increased by parallelisation using multiple cores.

The raw target signal 11 was processed to produce a compressed sequence of target symbols 14.

The method of FIG. 1 was applied separately to each read of the target signal 11 using the following parameters.
1. The input sample data was normalised by multiplying by a constant and then subtracting a constant so that it had median value zero and median absolute deviation 1.
2. A median filter with window size 5 was applied.
3. The data were segmented in step T1 into the series of signal levels 12. Moving from sequentially through the vector of (median-filtered) samples of the target signal 11, a new level is begun whenever the difference between the next sample value and the median of all samples in the current level is more than 0.2.
4. The current value for each signal level was estimated as the median of all the sample values contained in the level.
5. Level values were then quantised in step T2 using the same method used for the sequence of reference symbols 23.
6. The sequence of target symbols 13 was run-length compressed in step T3 to provide the compressed sequence of target symbols 14.
7. In step A1, the compressed sequence of target symbols 14 was mapped against the compressed sequence of reference symbols 24.

All these steps were implemented in the programming language python, step 7 using the open-source python library ‘mappy’ which provides an interface to minimap. Using 40 cores on the same machine for a direct comparison with base calling, the time taken for steps 1-7 to be carried out on all the reads was 58 seconds.

Thus, the total time for performance of the method was a couple of minutes, which is a significant saving on the comparative method that takes more than 3 hours for the basecalling of the target signal 11, as described above.

The locations of the reads in the reference sequence 22, as derived from the mapping in step A1 was compared with the locations derived from mapping of the basecalls. The locations derived from the method of FIG. 1 overlapped with the basecall-derived locations in 99.7% of the reads (4986 out of 5000).

1. A method of determining a relationship (30) between a target sequence of polymer units in a target polymer (10) and a reference sequence of polymer units, wherein the method comprises:
receiving a measured target signal (11) comprising signal levels measured by a measurement system from parts of the target polymer (10) ordered along the target sequence;
segmenting the measured target signal (10) into segments and deriving a sequence of target signal symbols (13), each target signal symbol representing a quantised signal level derived from the signal levels of a respective segment (steps T1, T2); and
using a sequence of reference signal symbols (23) representing quantised signal levels of a sequence of modelled reference signal levels predicted by a measurement system model to be measured from the reference sequence of polymer units by the measurement system, comparing (step A1) the sequence of target signal symbols (13) with the sequence of reference signal symbols (23) to determine the relationship (30) between the target sequence and the reference sequence.

2. A method according to claim 1, wherein (step T3) the sequence of target signal symbols (13, 14) are run-length compressed before the step of comparing (step A1) the sequence of target signal symbols (13) with the sequence of reference signal symbols (23).

3. A method according to claim 1 or 2, wherein (step R3) the sequence of reference signal symbols (23, 24) are run-length compressed before the step of comparing (step A1) the sequence of target signal symbols (13) with the sequence of reference signal symbols (23).

4. A method according to any one of the preceding claims, wherein the step of segmenting the measured target signal into segments (step T1) comprises detecting transitions in the signal level of the measured target signal (11) and segmenting the measured target signal (11) into segments defined between the transitions.

5. A method according to claim 4, wherein the step of segmenting the measured target signal (step T1) into segments further comprises smoothing the measured target signal (11) prior to detecting transitions in the signal level of the measured target signal (11).

6. A method according to claim 5, wherein the step of smoothing the measured target signal (11) is performed by total-variation de-noising.

7. A method according to any one of the preceding claims, wherein the step of deriving a sequence of target signal symbols (13) comprises:
deriving an average signal level (12) from the signal levels of each segment (step T1);
deriving the target signal symbols by quantising the average signal levels in respect of each segment (step T2).

8. A method according to any one of the preceding claims, wherein the target signal symbols (13) and the reference signal symbols (14) represent quantised signal levels with a quantisation providing equal populations in each symbol.

9. A method according to any one of the preceding claims, further comprising deriving the sequence of reference signal symbols (23) from the reference sequence (22) (step R2), the modelled reference signal levels of the reference signal symbols (23) being predicted by the measurement system model to be measured from the reference sequence (22) by the measurement system.

10. A method according to claim 9, further comprising:
receiving a measured reference signal (21) comprising signal levels measured by a measurement system from parts of a reference polymer (20) ordered along the reference sequence; and
estimating the reference sequence from the measured reference signal using the measurement system model (step R1), the reference sequence (22) used in the step of deriving the sequence of reference signal symbols (23) from the reference sequence being the estimated reference sequence (22).

11. A method according to claim 9, wherein the reference sequence is stored in a memory.

12. A method according to any one of the previous claims, wherein the reference sequence of polymer units corresponds to the entirety or a region of a reference polymer.

13. A method according to any one of the previous claims, wherein the target sequence of polymer units corresponds to the entirety or a region of the target polymer.

14. A method according to any one of the previous claims, wherein the reference sequence of polymer units corresponds to a region of a reference polymer that is the same polymer as the target polymer.

15. A method according to any one of the preceding claims, wherein the step of comparing (step A1) the sequence of target signal symbols (13) with the sequence of reference signal symbols (23) is performed using a weight matrix that takes into account differences between the quantised levels represented by the target signal symbols (13) and the reference signal symbols (23).

16. A method according to any one of the preceding claims, wherein the determined relationship comprises an alignment between the target sequence and the reference sequence.

17. A method according to any one of the preceding claims, further comprising determining if all or part of the reference sequence (22) is present or absent in the target sequence (step A2) from the determined relationship (30) between the target sequence and the reference sequence.

18. A method according to any one of the preceding claims, wherein the method is repeated with plural reference sequences (22).

19. A method according to claim 18, wherein the plural reference sequences correspond to plural different reference polymers or to different regions of the same reference polymer.

20. A method according to claim 18 or 19, further comprising determining if all or part of any of the reference sequences (22) is present or absent in the target sequence (step A2) from the determined relationship between the target sequence and the reference sequence.

21. A method according to any one of the preceding claims, wherein the determined relationship comprises a measure of similarity between the target sequence and the reference sequence.

22. A method according to claim 21, wherein the determined relationship is used to reject the target polymer in favour of measuring another target polymer.

23. A method according to any one of the preceding claims, wherein the polymer is a polynucleotide, and the polymer units are nucleotides.

24. A method according to any one of the preceding claims, wherein the measurement system comprises a nanopore and the measured target signal (11) comprises signal levels measured by the measurement system during translocation of the polymer with respect to the nanopore.

25. A method according to claim 24, wherein the nanopore is a protein pore.

26. A method according to claim 24 or 25, further comprising the step of ejecting the polymer from the nanopore during translocation depending upon the measure of similarity.

27. A method according to any one of the preceding claims, wherein the signal levels representing one or more of: ionic current, impedance, a tunnelling property, a field effect transistor voltage and an optical property.

28. A method according to any one of the preceding claims, further comprising deriving the measured target signal by measuring the signal levels by the measurement system (step TM).

29. A computer program capable of execution by a computer apparatus and configured, on execution, to cause the computer apparatus to perform a method according to any one of claims 1 to 27.

30. A computer-readable storage medium storing a computer program according to claim 29.

31. An analysis apparatus arranged to determining a relationship between a target sequence of polymer units in a target polymer (10) and a reference sequence of polymer units, the analysis apparatus being arranged to receive a measured target signal (11) comprising signal levels measured by a measurement system from parts of the target polymer (10) ordered along the target sequence, wherein the analysis apparatus comprises:
a target signal processing functional block (steps T1, T2) arranged to segment the measured target signal (10) into segments, and to derive a sequence of target signal symbols (13), each target signal symbol representing a quantised signal level derived from the signal levels of a respective segment; and
an analysis functional block (step A1) arranged to use a sequence of reference signal symbols (23) representing quantised signal levels of a sequence of modelled reference signal levels predicted by a measurement system model to be measured from the reference sequence of polymer units by the measurement system, and to compare the sequence of target signal symbols (13) with the sequence of reference signal symbols (23) to determine the relationship (30) between the target sequence and the reference sequence.
